(19) United States
(12) Patent Application Publication
(10) Pub. No.: US 2009/0287111 A1
(43) Pub. Date: Nov. 19, 2009

(54) ENDOSCOPE COLLECTION VESSEL

(75) Inventors: Christopher J. Kaye, Concord, OH (US); Gary E. Mann, Springfield, OH (US); Dean J. Secrest, Concord, OH (US)

Correspondence Address: CALFEE HALTER & GRISWOLD, LLP, 800 SUPERIOR AVENUE, SUITE 1400, CLEVELAND, OH, 44114, US

(73) Assignee: US ENDOSCOPY GROUP, INC., Mentor, OH (US)

(21) Appl. No.: 12/467,726

(22) Filed: May 18, 2009

Related U.S. Application Data
(60) Provisional application No. 61/053,981, filed on May 16, 2008.

(51) Int. Cl. A61B 10/04; A61B 1/012

(52) U.S. Cl. 600/562; 600/101

(57) ABSTRACT

A device for use with an endoscope is disclosed. The device includes a body that defines a vessel for collecting fluid that may escape through the port and/or valve of the endoscope. The body includes a bottom and a side wall. The bottom and the side wall form a portion of a cradle surrounding the port of the endoscope. The bottom includes a portion configured to mate with the endoscope adjacent the port. The side wall is configured to partially wrap around the port of the endoscope. Further, the body of the device may be shaped to cover at least the neck portion of the port and may include an aperture. The aperture allows access to the port and facilitates attachment of the valve. Absorbent material may be used in the collection vessel to absorb the fluid. The device may also include a wire anchor for attaching a guide wire.

### CROSS-REFERENCE TO RELATED APPLICATION

This application claims the benefit of U.S. Provisional Patent Application Ser. No. 61/053,981, entitled “ENDOSCOPE COLLECTION VESSEL” and filed May 16, 2008, the entire contents of which are incorporated herein by reference, to the extent that they are not conflicting with the present application.

### FIELD OF THE INVENTION

This application relates to an endoscopic medical device and more particularly to a collection vessel for use with a port of an endoscope during endoscopic medical procedures.

### BACKGROUND OF THE INVENTION

Endoscopic retrograde cholangiopancreatography (ERCP) enables a physician to diagnose problems in the liver, gallbladder, bile ducts, and pancreas. ERCP can be used to diagnose and treat conditions of the bile ducts, including gallstones, inflammatory strictures (scars), leaks (from trauma and surgery), and cancer. ERCP combines the use of x rays and an endoscope. Through the optical features of the endoscope, the physician can see the inside of the stomach and duodenum, and inject dyes into the ducts in the biliary tree and pancreas so they can be seen on x rays.

The endoscope is generally inserted through the mouth, down the esophagus, into the stomach and through the pylorus into the duodenum, the first portion of the small intestines. The ampulla of Vater is located in the duodenum and is the opening of the common bile duct and pancreatic duct. The region can be directly visualized with the endoscopic camera while various procedures are performed. A plastic catheter or cannula is inserted through a valve attached to the instrument port, or biopsy port, at the proximal end of the endoscope. The catheter travels through the valve and instrument channel of endoscope, out the distal end of the endoscope, and through the ampulla.

Further, a channel, e.g., a U-shaped open channel or closed channel, of the catheter may contain a guide wire that is passed into the bile duct. The guide wire may be used to guide other instruments down the instrument channel of the endoscope, through the ampulla, and into the bile duct. For example, if the exam shows a gall stone or narrowing of the ducts, the physician can insert instruments into the scope and down the guide wire to remove or relieve the obstruction. Also, tissue samples (biopsy) can be taken for further testing. In order for the guide wire to be used with other instruments, the catheter must be removed, or peeled, from the guide wire and taken out of the instrument channel of the endoscope. Further, the guide wire generally needs to be held, locked, or otherwise controlled at the proximal end of the endoscope during removal of the catheter and/or insertion of other instruments. The anchored guide wire acts as a foundation for the repetitive attachment and removal of instruments to the guide wire during various endoscopic procedures.

The manipulation of the catheter, guide wire, or other instruments creates a lateral force on the opening in the valve attached to the instrument port of the endoscope, especially when the catheter is being removed from the guide wire. As such, fluid trapped inside the instrument channel of the endoscope may escape through the opening. Because the guide wire holds the bile duct open, capillary action will allow bile to move up the wire and instrument channel of the endoscope and escape, or seep, through the opening in the valve. The amount of escaped fluid will depend on various factors, including for example the length of the procedure, or the type of procedure.

It should be noted that fluid may escape from the instrument channel of the endoscope during other endoscopic procedures besides ERCP and whether or not a guide wire is used during the procedure, for example, colonic screening or EGD.

### SUMMARY OF THE INVENTION

In an illustrated embodiment of the invention, a collection device is disclosed. The collection device is for use with a port of an endoscope during endoscopic medical procedures.

A collection device for use with an endoscope is disclosed. In one embodiment, the collection device includes a body that defines a vessel for collecting fluid that may escape through the port and/or valve of the endoscope. The body includes a bottom and a side wall. The bottom and the side wall form a portion of a cradle surrounding the port of the endoscope. The bottom includes a portion configured to mate with an outer surface of the endoscope adjacent the port. The side wall is configured to partially wrap around the port of the endoscope. Further, the body of the collection device may be shaped to cover at least the neck portion of the port and may include an aperture. The aperture allows access to the port and facilitates attachment of the valve to the port. Absorbent material may be used within and/or without the collection vessel to absorb the fluid. The collection device may also include a wire anchor for attaching a guide wire. The collection vessel may also include an opening to accommodate a currently available locking device.

Further features and advantages of the invention will become apparent from the following detailed description made with reference to the accompanying drawings.

### BRIEF DESCRIPTION OF THE DRAWINGS

FIG. 1 is a top front perspective view of a collection device according to an embodiment of the invention;

FIG. 2 is a bottom rear perspective view of the collection device of FIG. 1;

FIG. 3 is a top view of the collection device of FIG. 1;

FIG. 4 is a perspective view of a portion of an exemplary endoscope;

FIG. 5 is a perspective view of the portion of the endoscope of FIG. 4 with the collection device of FIG. 1 attached;

FIG. 6 is a perspective view of the portion of the endoscope of FIG. 4 with the collection device of FIG. 1 attached, wherein a valve is attached to an instrument port of the endoscope;

FIG. 7 is a side view of the endoscope of FIG. 4 with the collection device of FIG. 1 attached, wherein the collection device, the valve, and a portion of the instrument port are shown in cross section taken along line 7-7 in FIG. 6;

FIG. 8 is a magnified view of a portion of FIG. 7;

FIG. 9 is a perspective view of the portion of the endoscope of FIG. 4 with the collection device of FIG. 1 attached, wherein a valve is attached to an instrument port of the endoscope and a guide wire is attached to a wire anchor; and

FIGS. 10A and 10B are perspective views of a collection device according to an embodiment of the invention.

### DETAILED DESCRIPTION OF THE INVENTION

The Detailed Description of the Invention merely describes preferred embodiments of the invention and is not intended to limit the scope of the invention in any way. Indeed, the claims as described by the specification are broader than and unlimited by the preferred embodiments, and the terms used have their full ordinary meaning.

A collection device for use with a port of an endoscope during endoscopic medical procedures is disclosed. For exemplary purposes only, the invention will be discussed in regard to a collection device designed for use with the instrument port, or biopsy port, of an endoscope. It should be apparent to others with ordinary skill in the art that the discussion and Figures included in this application are by way of example only, and that the invention can be utilized with other ports and other endoscopic devices having a wide variety of structures, shapes, or purposes.

The exemplary collection device includes a body portion that defines a vessel for collecting fluid that may escape through the instrument port or valve of the endoscope. The body portion includes a curved wall that defines at least one concave side wall of the vessel. Further, the body portion includes a bottom that defines the bottom of the vessel. At least a portion of the bottom is curved such that the collection device may at least partially wrap around the main body of the endoscope. Extending from the bottom of the body portion is a protrusion or neck shaped to at least partially cover the neck of the instrument port of the endoscope. The neck of the body portion includes an aperture at the top to allow access to the instrument port and attachment of a valve.

In some embodiments, the body portion of the collection device includes at least one back wall connecting at least a portion of the bottom to at least a portion of the side wall. The body portion of the collection vessel may also include an opening to accommodate a currently available locking device. The body portion of the collection device is generally made from a single piece of molded plastic. However, the body portion may be made by any suitable method or from any suitable material known in the art, such as for example, a flexible elastomer molded component or a bag (such as a polyethylene bag) that can be mounted under the instrument port to collect fluids. The bag or flexible elastomer may contain a rigid deck that can assist in mounting to the deck below the instrument port. Further, the body portion of the collection device may be made from more than one piece of material. The vessel of the collection device may be sized to accommodate, or hold, various amounts of fluid, such as for example, 10-100 cc of fluid.

As stated, at least a portion of the bottom of the body portion is curved such that the collection device may at least partially wrap around the main body of the endoscope. As such, the collection device may press fit or snap onto the main body of the endoscope. The neck of the body portion covering the neck of the instrument port inhibits the collection device from rotating relative to the endoscope. Further, the top of the neck of the body portion is pinched between the valve attached to the instrument port and the neck of the instrument port, prohibiting the collection device from coming off the endoscope. In some embodiments, material, such as rubber or elastomer, may be attached to the outside surface of the curved bottom such that the collection device grips the endoscope. In other embodiments, a strap may be used to hold the collection device in place on the endoscope. Further, the collection device may be sized to accommodate various brands, types, and sizes of endoscopes and instrument ports.

Absorbent material, such as for example, cloth, foam, or a sponge-like material, may be used in the vessel to absorb the fluid that may escape through the instrument port or valve of the endoscope. For example, the absorbent material may be shaped to occupy at least a portion of the volume of the vessel bounded by the bottom, neck, and side wall of the body portion. In addition, absorbent material may be disposed on the outside of the body portion of the collection device to absorb fluids and to dry the physician's hands. In some embodiments, the absorbent material is attached to the body portion of the collection device, for example with an adhesive.

Further, the collection device may include features to hold the absorbent material in place. These features may be attached to or integrally formed with the body portion of the collection device. For example, in one embodiment of the invention, ridges on the side wall, bottom, and neck of the body portion engage or pinch the absorbent material and hold it in place within the vessel. In other embodiments, a clamping component is attached via a hinge, such as for example, a living hinge or pin, to the body portion of the collection device. The clamping component may include a spike, pin, or bar that holds the absorbent material in place within the vessel of the collection device. In these embodiments, the clamping component may be rotated to pinch the absorbent material against at least one of the side wall, bottom, or neck of the body portion. The clamping component may also be locked, such as for example, with a snap fit, to prohibit the absorbent material from coming out of the vessel.

The collection device may also include a wire lock for anchoring the guide wire. The guide wire is generally wrapped around tabs, prongs, flanges, or cleats on the wire lock and/or threaded through holes in the wire lock to prohibit the end of the wire from moving. In some embodiments, the wire lock is a separate component that can be removably attached to the body portion of the collection device, before or after the biopsy valve has been attached to the instrument port. For example, a portion of the wire lock may be formed to attach to, such as with a snap fit, a corresponding structure or opening in the body portion. In other embodiments, the wire lock is integrally formed with the body portion, for example extending from the side wall of the body portion. In still other embodiments, the wire lock is movably attached to the collection device. For example, the wire lock may be rotated relative to the collection device and locked into place.

The collection device may also include a drain, or suction port, for removal of the fluid from the vessel. For example, in one embodiment, the collection device includes a tube, or hose, extending from an aperture in the side wall or bottom of the body portion. Fluid from the vessel travels through the aperture and out the tube. The tube may have a plug or valve in the end to control the drainage of the fluid. Further, the collection device may include a suction port for suctioning the fluid from the vessel, utilizing the suction controls of the endoscope or by a separate suction source with independent on/off control.

Further, the valve attached to the instrument port of the endoscope may be connected, either by a fixed or removable connection, to a line, or tube, extending into the vessel of the collection device. The line may be anchored to the body portion of the collection device, such as for example, friction fit within a ring integrally molded with the body portion of the vessel. When suction is applied to the control head of the endoscope (i.e., scope suction), accumulated fluid in the collection device may be drawn up through the line, through the instrument port, and out the control head of the endoscope. Further, a one-way valve, such as a check or flapper valve, may be placed in the line, such as for example, at the connection of the line and the valve. The one-way valve would open during scope suction to allow the fluid to be suctioned out of the vessel of the collection device and out the control head of the endoscope. When the suction is turned off or has ceased, the one-way valve would close to prohibit any accumulated fluid in the device from traveling through the line and into the instrument port and channel of the endoscope. The one-way valve would also prevent any irrigation fluid introduced through the top of the biopsy valve, or at the proximal end of the endoscope, from traveling down the tube and into the vessel of the collection device.

The collection device may also include anchors to assist with holding the collection device in place on the main body of the endoscope. The anchors may be integrally formed with the collection device. For example, two anchors may extend from the side wall of the collection device. A strap may attach to the first anchor, wrap around the main body portion of the endoscope, and attach to the second anchor, thus holding the collection device in place on the endoscope.

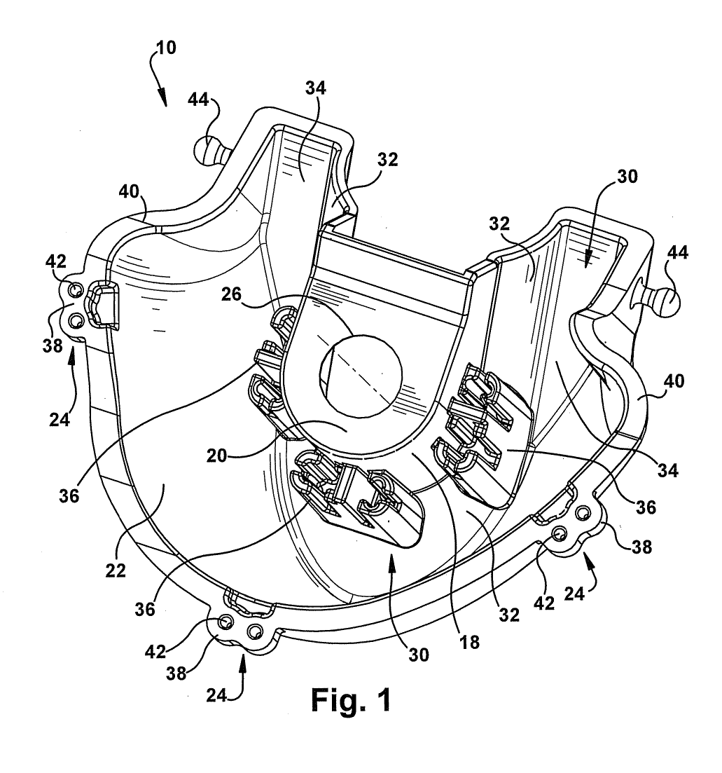

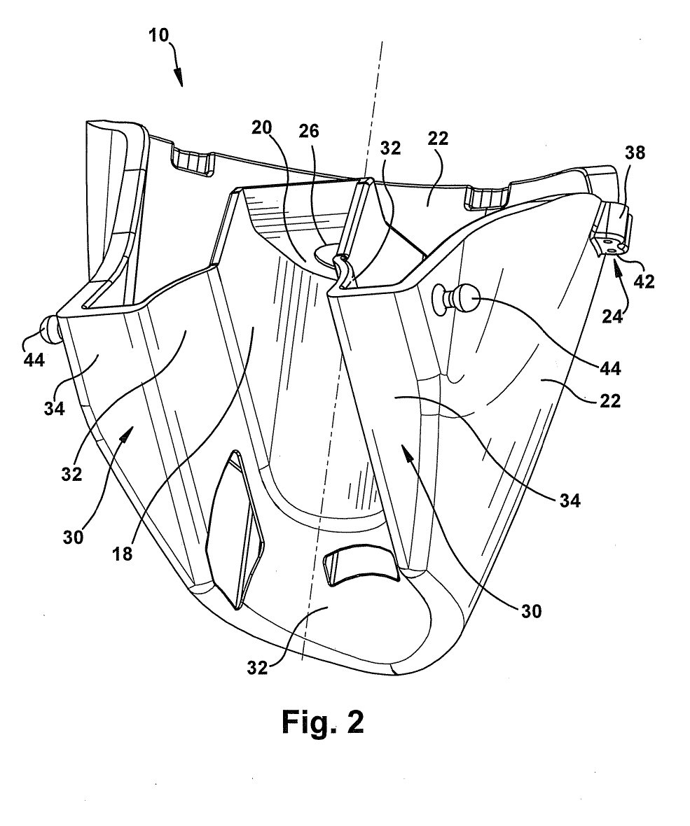

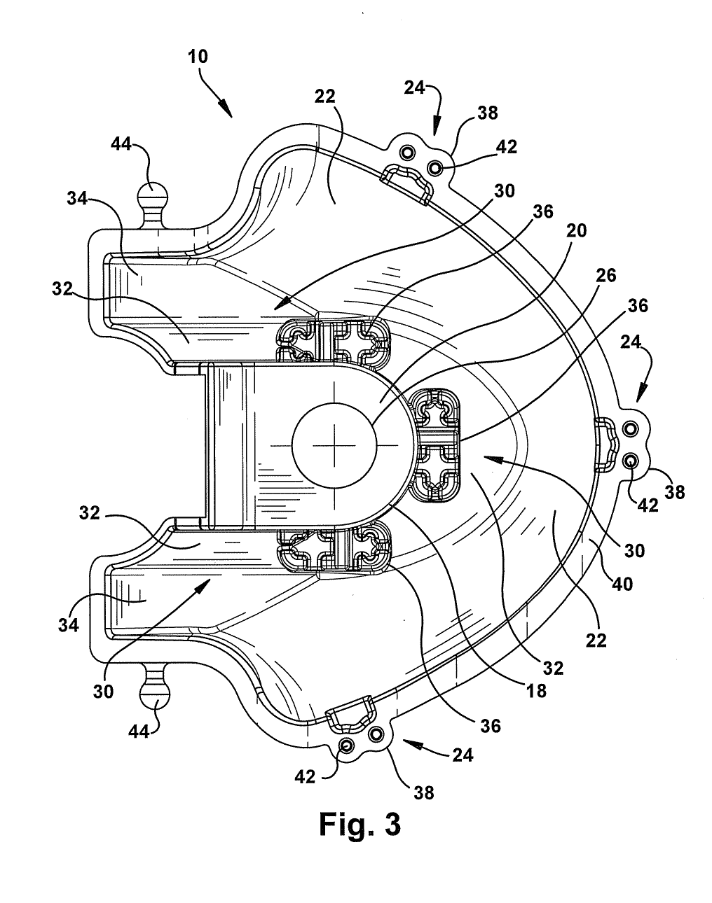

Referring now to the drawings, FIGS. 1-3 are various views of an exemplary collection device 10 according to an embodiment of the invention. The collection device 10 includes a body portion that defines a vessel for collecting fluid that may escape through an instrument port and/or valve of an endoscope. The vessel may be sized to accommodate, or hold, various amounts of fluid, such as for example, 10-100 cc of fluid. Further, the collection device 10 may be sized to accommodate various brands, types, and sizes of endoscopes and instrument ports.

The body portion of the collection device includes: a curved wall that defines at least one concave side wall 22 of the vessel; a bottom 30 that defines the bottom of the vessel; and a protrusion or neck 18 extending from the bottom 30 shaped to at least partially cover the neck of the instrument port. The body portion is made from a single piece of molded plastic. However, the body portion may be made by any suitable method or from any suitable material known in the art, such as for example, a flexible elastomer molded component. Further, the body portion may be made from more than one piece of material.

As shown in FIGS. 1-3, the bottom 30 includes a curved portion 32 and two substantially planar portions 34. The curved portion 32 of the bottom 30 is configured to mate with the body of the endoscope by wrapping at least partially around the outer surface of the endoscope. The curved portion 32 may be configured to press fit or snap onto the outer surface of the endoscope such that the collection device 10 is held in place relative to the endoscope. In some embodiments, material, such as rubber or elastomer, may be attached to the outer surface of the curved portion 32 such that the collection device 10 grips the endoscope. In other embodiments, the curved portion 32 may be attached to the outer surface of the endoscope by other various means, for example, with a fastener or adhesive.

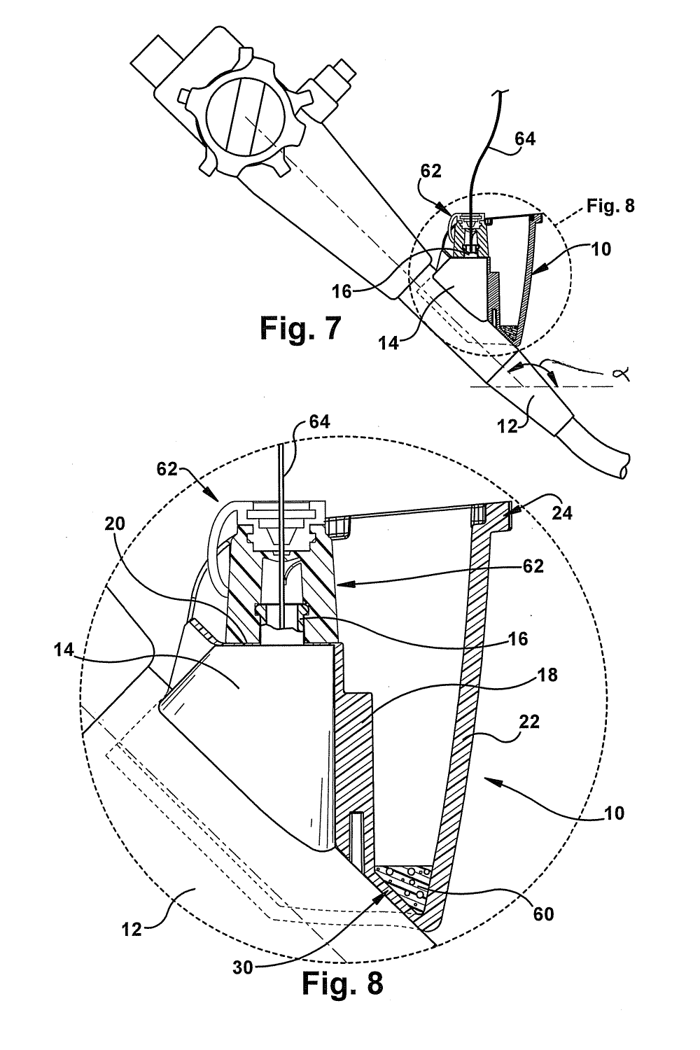

Each planar portion 34 of the bottom 30 extends between the curved portion 32 and at least a portion of the side wall 22 on each side of the collection device 10. Thus, the planar portions 34 permit at least a portion of the side wall 22 to be spaced from the body of the endoscope. This spacing provides room for the operator to access the instrument port of the endoscope. Further, when the collection device 10 is positioned for use (as shown in FIGS. 7 and 8), the planar portions 34 of the bottom 30 act as back walls that prevent fluid from escaping out of the collection device 10. In some embodiments, the bottom of the collection device does not include a planar portion. Thus, in these embodiments, the curved portion intersects directly with the side wall of the collection device. In other embodiments, the body portion of the collection device may include at least one separate back wall connecting at least a portion of the bottom (e.g., the curved portion and the planar portion) to at least a portion of the side wall.

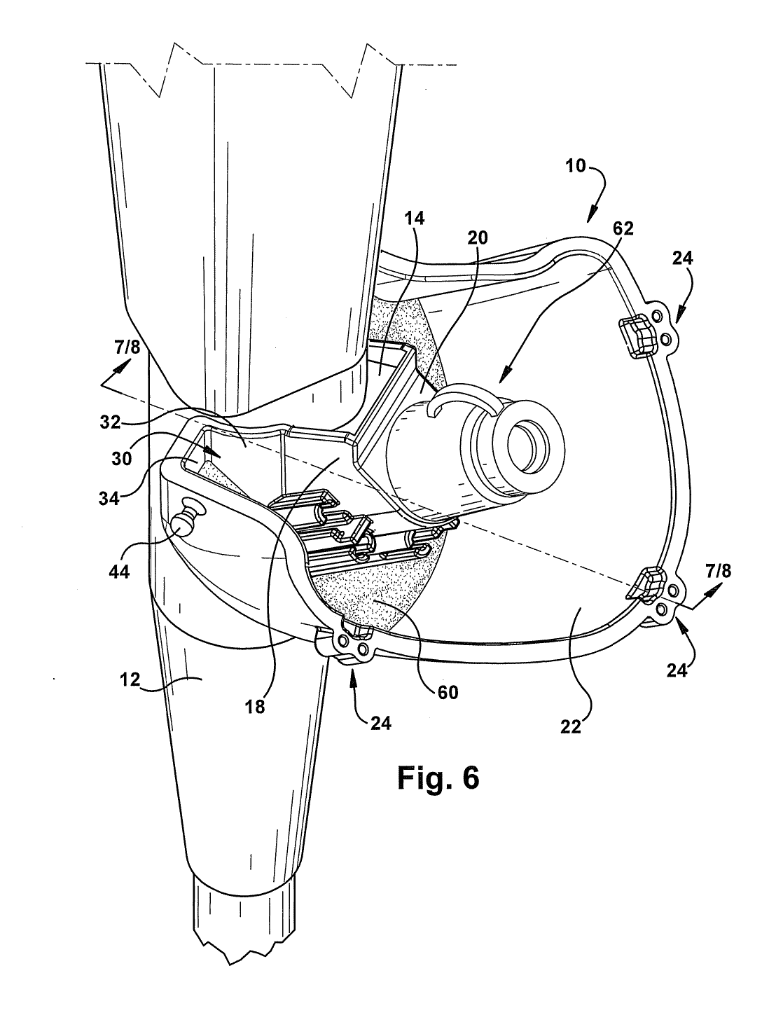

The side wall 22 of the body portion is configured to wrap at least partially around the instrument port of the endoscope. The side wall 22 and the bottom 30 are designed such that the vessel of the collection device 10 creates a cradle surrounding the instrument port when the endoscope is used at angles conventionally used in endoscopic procedures (e.g., FIGS. 6 and 7). As shown in FIGS. 1-3, the side wall 22 is a curved, concave wall that intersects the curved portion 32 and planar portions 34 of the bottom 30. The side wall 22 extends from one planar portion 34, around the instrument port, to the other planar portion 34. However, the side wall 22 may be various shapes and configurations capable of forming at least a portion of a cradle surrounding the instrument port when the endoscope is used at angles conventionally used in endoscopic procedures. For example, the side wall may include three planar portions that at least partially surround the instrument port of the endoscope.

As shown in FIGS. 1-3, the neck 18 of the body portion extends from the curved portion 32 of the bottom 30. The neck 18 is shaped to at least partially cover the neck of the instrument port. The neck 18 inhibits the collection device 10 from rotating relative to the endoscope, and also inhibits axial movement of the collection device along the endoscope. An aperture 26 extending through a top face 20 of the neck 18 permits access to the instrument port of the endoscope and the attachment of the valve. Further, when the collection device 10 is in use, the top face 20 of the neck 18 is pinched between the valve attached to the instrument port and the neck of the instrument port, prohibiting the collection device from coming off the endoscope. In some embodiments, the neck does not include a top face with an aperture, but instead only extends partially up the neck of the endoscope. Further, in other embodiments, the body portion does not include a neck. In these embodiments, the instrument port extends through an opening in the curved portion of the bottom. In these and other embodiments, the collection device is also inhibited from rotating relative to the endoscope or moving axially along the endoscope.

As shown in FIGS. 1-3, connectors 36 extend as protrusions from the curved portion 32 of the bottom 30. The connectors 36 are shaped and configured to mate with various components, such as an insertable wire lock. Although not shown, these components are configured to be removably connected to the connectors 36, such as with a snap or press fit. As shown in FIGS. 1-3, three connectors 36 extend from the curved portion 32 of the bottom 30. The connectors 36 are spaced around the neck 18 such that the components may be positioned in various locations relative to the instrument port of the endoscope (e.g., right side, left side, etc.). However, the collection device may include more or less connectors positioned in various locations on the collection device.

As shown in FIGS. 1-3, three wire anchors 24 for routing a guide wire are integrally formed with the body portion of the collection device 10. Each wire anchor 24 includes a flange 38 that extends outward from an edge 40 of the side wall 22. Each flange 38 includes two openings 42. The guide wire may be wrapped around the flange 38 of the wire anchor 24 and/or threaded through the openings 42 to prohibit the end of the wire from moving. The wire anchors may be a variety of shapes and configurations, such as tabs, prongs, or cleats. Further, the collection device may include more or less wire anchors positioned in various locations on the collection device.

As shown in FIGS. 1-3, the body portion of the collection device includes two mounts 44 extending from the side wall 22. The mounts 44 are designed to assist with holding the collection device 10 in place on the body of the endoscope. As shown, the mounts 44 are integrally formed with the side wall 22; although, the mounts may be attached to the side wall, such as with a fastener or adhesive. The mounts 44 are configured such that a strap (not shown) may attach to one mount, wrap around the body portion of the endoscope, and attach to the other mount. Thus, the strap holds the collection device 10 in place on the endoscope. The mounts may be various shapes and configurations, such as a pin, a latch, a buckle, a clamp, hook and lock style mechanical fasteners, or the like. Further, the strap may be adjustable to accommodate various brands, types, and sizes of endoscopes.

Absorbent material, such as for example, cloth, foam, or a sponge-like material, may be used inside or outside the vessel to absorb the fluid that may escape through the instrument port or valve of the endoscope. For example, the absorbent material may be shaped to occupy at least a portion of the volume of the vessel bounded by the bottom 30, neck 18, and side wall 22 of the body portion. In addition, the absorbent material may be attached to the outside of the side wall 22 of the body portion. In some embodiments, the absorbent material is attached to the body portion of the collection device, for example, with an adhesive. The absorbent material may be attached to one or more of the bottom 30, neck 18, and side wall 22 of the body portion, and the shape and depth of the absorbent material may vary.

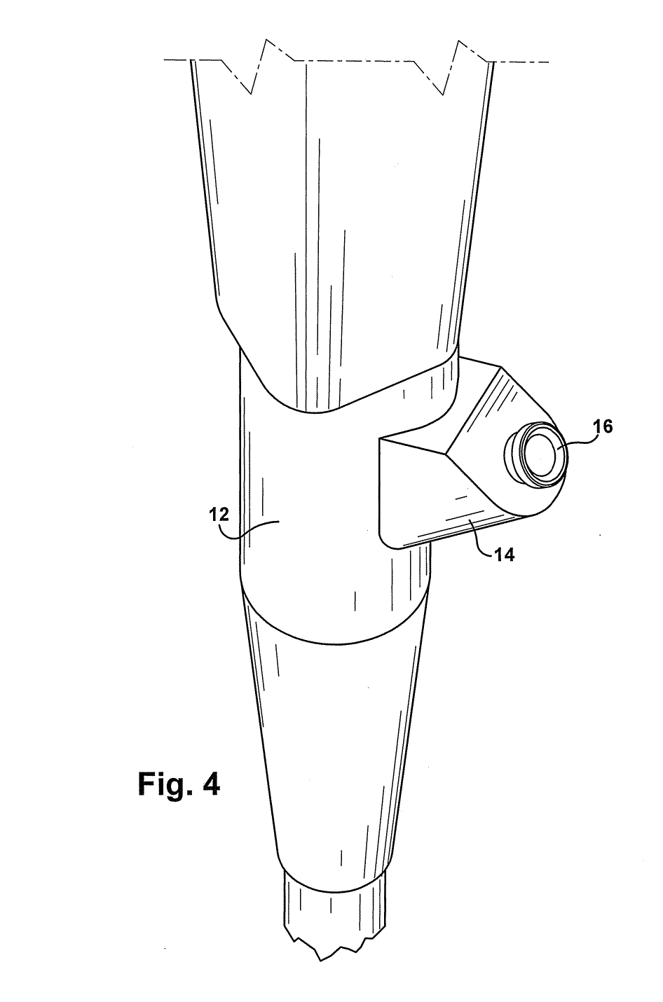

FIG. 4 is a perspective view of an exemplary endoscope. The exemplary endoscope includes a main body 12 and an instrument port 16, or biopsy port, having a neck portion 14 extending from the main body.

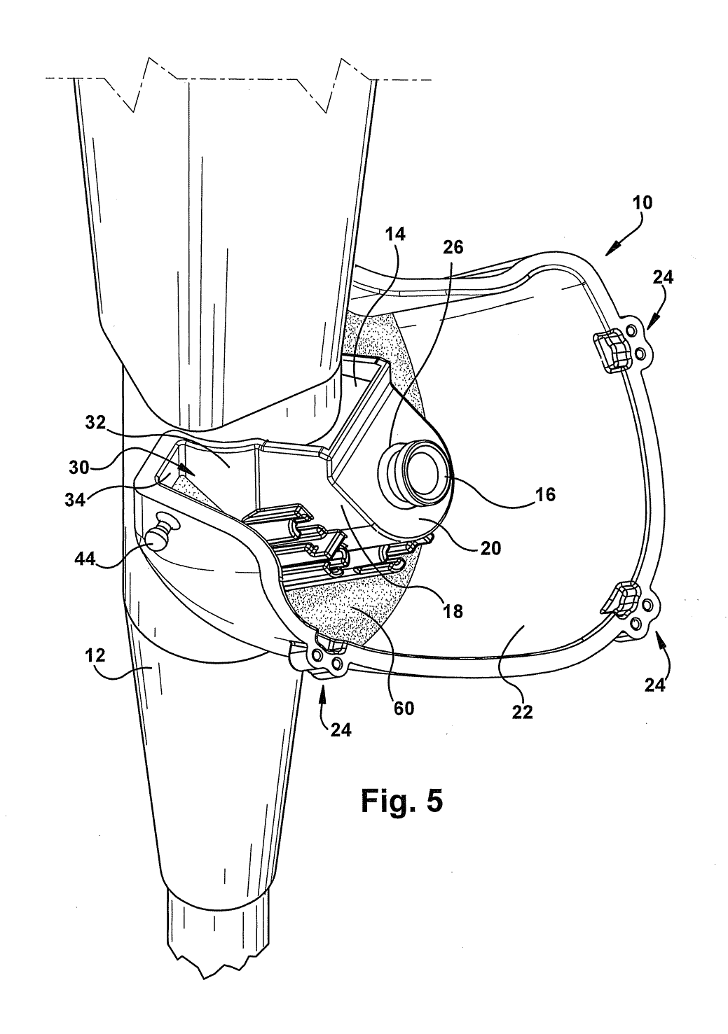

FIG. 5 is a perspective view of the collection device 10 of FIGS. 1-3 attached to the main body 12 of the endoscope. The body portion of the collection device 10 defines a vessel for collecting fluid that may escape through the instrument port 16 of the endoscope. As shown, the curved portion 32 of the bottom 30 wraps partially around the outer surface of the main body 12 of the endoscope. The side wall 22 of the body portion wraps partially around the instrument port 16 of the endoscope. The side wall 22 and the bottom 30 are designed such that the vessel of the collection device 10 creates a cradle surrounding the instrument port 16. Further, the neck 18 of the body portion substantially covers the neck portion 14 of the endoscope extending from the main body 12. The neck 18 of the body portion inhibits the collection device 10 from rotating relative to the endoscope, or from movement axially along the endoscope. The aperture 26 extending through the top face 20 of the neck 18 permits access to the instrument port 16 of the endoscope and the attachment of a valve. Absorbent material 60 is shown occupying at least a portion of the volume of the vessel bounded by the bottom 30, neck 18, and side wall 22 of the body portion.

FIG. 6 is a perspective view of the collection device 10 of FIGS. 1-3 attached to the main body 12 of the endoscope, wherein a valve 62 is attached to the instrument port 16 of the endoscope. As shown, the top face 20 of the neck 18 of the body portion is pinched between the valve 62 attached to the instrument port 16 and the neck portion 14 of the endoscope extending from the main body 12. Pinching the neck 18 of the body portion between the valve 62 and the neck portion 14 of the endoscope prohibits the collection device 10 from coming off the endoscope.

FIGS. 7 and 8 are side views of the collection device 10 of FIGS. 1-3 attached to the main body 12 of the endoscope, wherein the collection device 10, the valve 62, and a portion of the instrument port 16 are shown in cross section taken along line 7-7 in FIG. 6. The endoscope is depicted at an exemplary angle α used in endoscopic procedures. This angle will vary in the use of the device 10 depending on many factors, such as for example, preference of the user, size and type of endoscope, and procedure being performed. Further, an endoscopic device 64 is shown extending through the valve 62 and the instrument port 16. As shown, the bottom 30, neck 18, and side wall 22 of the collection device 10 define a vessel for collecting fluid that may escape through the instrument port 16 and/or the valve 62. The side wall 22 and the bottom 30 of the collection device 10 are designed such that the vessel of the collection device creates a cradle surrounding the instrument port 16 and the valve 62. Further, absorbent material 60 occupies at least a portion of the volume of the vessel bounded by the bottom 30, neck 18, and side wall 22. The absorbent material 60 may be attached to the collection device 10, such as with an adhesive. For example, the absorbent material 60 may be attached to any one or more of the bottom 30, neck 18, and side wall 22 of the collection device 10. Further, the absorbent material 60 may line any one or more of the bottom 30, neck 18, and side wall 22 of the collection device 10.

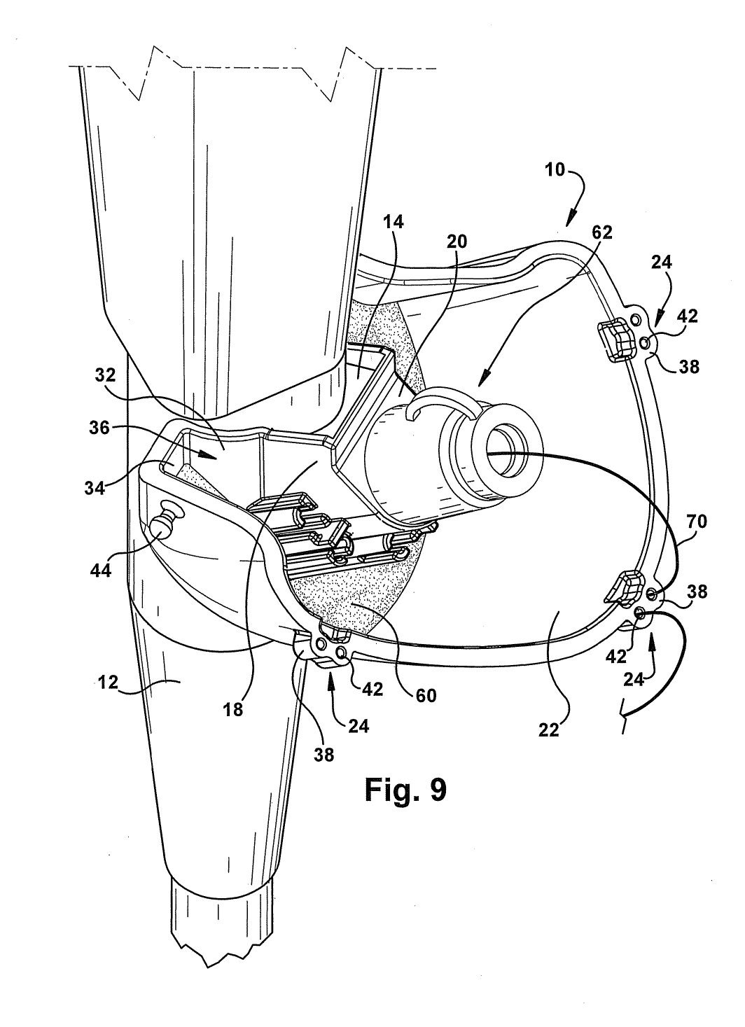

FIG. 9 is a perspective view of the collection device 10 of FIGS. 1-3 attached to the main body 12 of the endoscope, wherein a valve 62 is attached to the instrument port 16 of the endoscope. A guide wire 70 is shown extending out of the valve 62. The guide wire 70 is attached to a wire anchor 24 of the collection device 10. As shown, the guide wire 70 is threaded through the openings 42 in the flange 38 of the wire anchor 24 to limit wire movement.

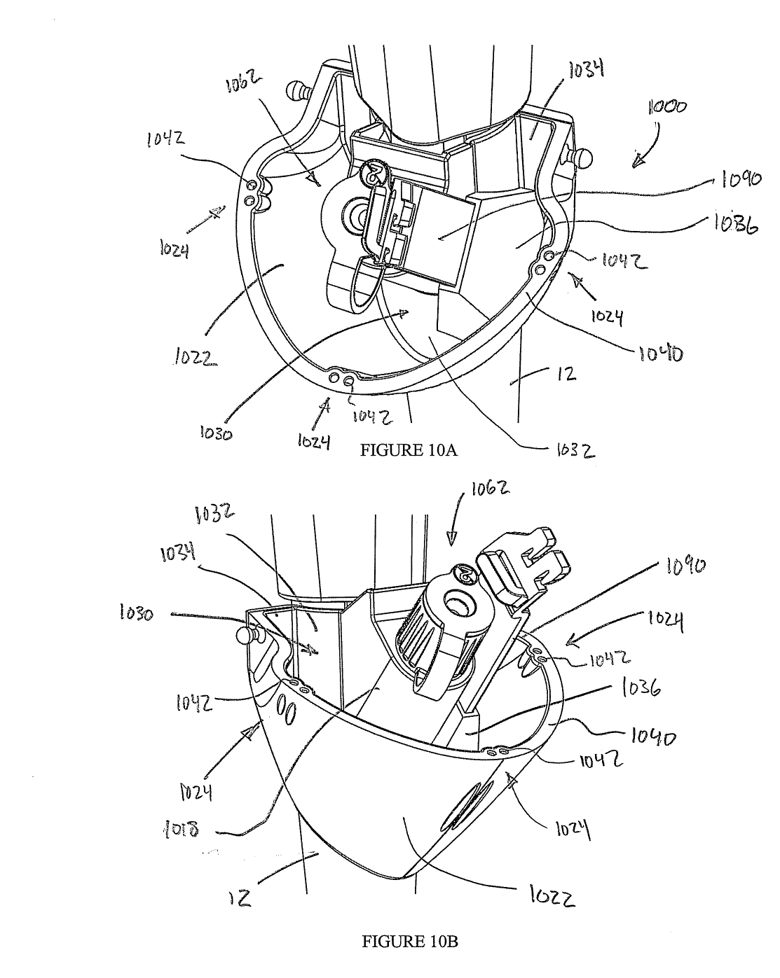

FIGS. 10A and 10B are perspective views of an exemplary collection device 1000 according to an embodiment of the invention. The collection device 1000 includes a body portion that defines a vessel for collecting fluid that may escape through an instrument port and/or valve of an endoscope. As shown, the collection device 1000 is attached to the main body 12 of the endoscope and a valve 1062 is attached to the instrument port (not shown) of the endoscope.

The body portion of the collection device includes: a curved wall that defines at least one concave side wall 1022 of the vessel; a bottom 1030 that defines the bottom of the vessel; and a protrusion or neck 1018 extending from the bottom 1030 shaped to at least partially cover the neck of the instrument port. The bottom 1030 includes a curved portion 1032 and two substantially planar portions 1034. The side wall 1022 of the body portion is configured to wrap at least partially around the instrument port of the endoscope. The side wall 1022 and the bottom 1030 are designed such that the vessel of the collection device 1000 creates a cradle surrounding the instrument port when the endoscope is used at angles conventionally used in endoscopic procedures. Further, the neck 1018 of the body portion extends from the curved portion 1032 of the bottom 1030.

As shown in FIGS. 10A and 10B, a connector 1036 extends from the curved portion 1032 of the bottom 1030. The connector 1036 is shaped and configured to mate with an insertable wire lock 1090. As shown, the connector 1036 comprises an opening for receipt of a stem of the insertable wire lock 1090. The stem may be removable, for example, press fit in the opening of the connection 1036. Further, various fasteners may be used to hold the wire lock 1090 in place relative to the connector 1036, such as a screw, pin, etc.

As shown in FIGS. 10A and 10B, three wire anchors 1024 for routing a guide wire are integrally formed with the body portion of the collection device 1000. Each wire anchor 1024 extends inward from an edge 1040 of the side wall 1022 and includes two openings 1042. The guide wire may be threaded through the openings 1042 to prohibit the end of the wire from moving.

While several embodiments of the invention has been illustrated and described in considerable detail, the present invention is not to be considered limited to the precise construction disclosed. Various adaptations, modifications and uses of the invention may occur to those skilled in the arts to which the invention relates. It is the intention to cover all such adaptations, modifications and uses falling within the scope or spirit of the claims filed herewith.

We claim:

1. A device for use with an endoscope having a port, comprising:
a body that defines a vessel for collecting fluid, the body having a bottom and a side wall; and
wherein the bottom and the side wall form a portion of a cradle surrounding the port of the endoscope, the bottom having a portion configured to mate with an outer surface of the endoscope adjacent the port, and the side wall configured to partially wrap around the port of the endoscope.

2. The device of claim 1, wherein the vessel is sized to accommodate between 10 cc and 100 cc of fluid.

3. The device of claim 1, wherein a curved portion of the bottom is configured to mate with the outer surface of the endoscope by partially wrapping around the outer surface.

4. The device of claim 3, wherein the curved portion snaps onto the outer surface of the endoscope such that the device is held in place relative to the endoscope.

5. The device of claim 3, wherein at least one planar portion of the bottom extends between the curved portion and a portion of the side wall.

6. The device of claim 5, wherein the bottom comprises a first planar portion and a second planar portion, each planar portion extending between the curved portion and a portion of the side wall, and wherein at least a portion of the side wall extends from the first planar portion, partially around the port of the endoscope, to the second planar portion.

7. The device of claim 1, wherein the side wall is a curved, concave wall that intersects the bottom of the body.

8. The device of claim 1, wherein the body further comprises a neck extending from the bottom and configured to at least partially cover a neck portion of the port.

9. The device of claim 8, wherein an aperture in a top face of the neck permits access to the port.

10. The device of claim 1, wherein the body further comprises at least one connector extending from the bottom.

11. The device of claim 1, wherein the body further comprises at least one wire anchor integrally formed with the side wall.

12. The device of claim 1, wherein the body further comprises at least one mount to hold the device in place on the endoscope.

13. The device of claim 1 further comprising an absorbent material shaped to occupy at least a portion of the volume of the vessel.

14. The device of claim 1 further comprising an absorbent material attached to a portion of the body.

15. A collection device for collecting fluid during an endoscopic procedure, comprising:
a body that defines a vessel for collecting fluid, the body having a bottom, a neck, and a side wall, the bottom having a curved portion wrapping partially around an outer surface of an endoscope adjacent a port of the endoscope, the neck extending from the bottom and configured to at least partially cover a neck portion of the port, and the side wall configured to partially wrap around the port; and
absorbent material shaped to occupy at least a portion of the volume of the vessel.

16. The device of claim 15, wherein the body further comprises a wire anchor integrally formed with the side wall.

17. The device of claim 15, wherein the absorbent material is attached to at least one of the bottom, the neck, and the side wall.

18. The device of claim 15, wherein an aperture in a top face of the neck permits access to the port.

19. A device for use with an endoscope having a port, comprising:
a body that defines a vessel for collecting fluid, the body having a bottom and a side wall; and
absorbent material shaped to occupy at least a portion of the volume of the vessel;
wherein the bottom and the side wall form a portion of a cradle surrounding the port of the endoscope, the bottom having a portion configured to mate with an outer surface of the endoscope adjacent the port, and the side wall configured to partially wrap around the port of the endoscope; and
wherein the body of the device limits the rotational movement of the device about the endoscope.

20. The device of claim 19, wherein the body of the device further comprises a neck that limits the rotational movement of the device about the endoscope.

21. The device of claim 19, wherein the body of the device limits the axial movement of the device along the endoscope.
